(19) United States
(12) Patent Application Publication
(10) Pub. No.: US 2024/0141254 A1
(43) Pub. Date: May 2, 2024

(54) 2,6,6-TRIMETHYL-NORPINAN-2-OL AS AN ODORIFEROUS SUBSTANCE

(71) Applicant: Symrise AG, Holzminden (DE)

(72) Inventors: Bernd HÖLSCHER, Halle (DE); Marc MANSFELD, Brevörde (DE); Julia AMOS, Eschershausen (DE); Lara-joy KLEINE-BENNE, Holzminden (DE); Theo BELMAS, Holzminden (DE)

(21) Appl. No.: 18/277,133

(22) PCT Filed: Mar. 9, 2021
(86) PCT No.: PCT/EP2021/055891

§ 371 Date: Aug. 14, 2023

(51) Int. Cl. C11B 9/00; A61K 8/34; A61Q 13/00; C07C 35/28

(52) U.S. Cl. CPC C11B 9/0046; A61K 8/34; A61Q 13/00; C07C 35/28

(57) ABSTRACT

The invention relates to the use of the compounds of formula (I) as an odoriferous substance.

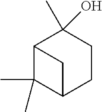

### FIELD OF THE INVENTION

The present invention relates to the use of 2,6,6-trimethyl-norpinan-2-ol, the compound of formula (I), as a fragrance. The present invention further relates to fragrance compositions, their use, and a method for imparting, modifying and/or enhancing certain scent notes.

### BACKGROUND OF THE INVENTION

The perfume industry constantly requires new fragrance creations. However, the creation of a new fragrance or perfume involves a number of challenges. For example, it is necessary to select a particular composition of a small number of fragrances from a nearly unlimited number of possible structures that are known from the state of the art in order to satisfy a specific demand of the market and to provide a product that corresponds with the specific profile required by the client. While suitable fragrances exhibit quite an advantageous performance, they may, however, develop unpleasant and therefore disadvantageous scent aspects in combination with other fragrances, which makes it difficult if not impossible to use them for the specific purpose.

A second important issue concerns the need to provide fragrance substance compositions which do not only correspond with a particular scent profile, but which also have what is referred to as secondary advantageous properties. In fact, many fragrance compositions known in the market have considerable disadvantages with respect to their application, for example, a poor solubility and a poor storage stability, but also errors with respect to subjective properties such as body, charism and the like. Furthermore, many known fragrance compositions require high dosages in order to achieve the desired scent result. Nowadays, a further requirement for fragrances is a high biological degradability as well as a dermatological and toxicological safety. As a consequence, a particularly high demand is to provide fragrances that both have a strong effect on other fragrances already in small dosages and change rather unpleasant scent impressions positively and/or enhance pleasant scent impressions.

Therefore, the task on which the present invention is based was to provide a new fragrance having the capability to improve positive and advantageous scent aspects of other fragrances and/or (simultaneously) to reduce, inhibit and/or mask any undesired, unpleasant scent aspects. Particularly, the new fragrance should also be characterized in that it improves the stability, solubility and total performance of other fragrances and that it reduces the required dosage. Finally, the fragrance compositions themselves should exhibit an excellent biological degradability and be safe both for humans and the environment.

### DESCRIPTION OF THE INVENTION

The primary task at hand is solved according to the invention by the use of the compounds of formula (I) (2,6,6-trimethyl-norpinan-2-ol, CAS No. 4948-28-1)

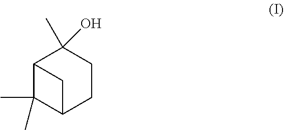

as a fragrance.

The compound of formula to be employed according to the invention may be present in any stereoisomeric form, or may be present as any mixture of stereoisomers.

What has been said herein about a compound of formula (I), particularly the advantages described herein, shall apply mutatis mutandis to a mixture of stereoisomers of the compound of formula (I) (see above) to be used or employed according to the invention.

In the event that are any unintentional discrepancies between the chemical name and the illustrated structural formula within the scope of this text, the illustrated structure formula shall prevail.

The compound of formula (I) has the most individual olfactory properties which clearly stand out from known fragrances, even excelling them. The suitability of the compounds of formula (I) as a fragrance has not been known to this date. Therefore, it is particularly surprising that it was possible to find a fragrance having valuable; interesting and complex olfactory properties in this well-researched field.

The compound of formula (I) is known within the fragrance industry under the name Pinanol (2,6,6-trimethyl-norpinan-2-ol) as an intermediate product from the process of production of linalool from pinene. As has been mentioned above, its suitability as a fragrance has not been known. In addition, no precise fragrance description has been available, however, according to the perfumers, the compound of formula (I) smells like pines, earthy and like rosemary, and, consequently, is rather unpleasant.

Consequently, the inventors made the surprising discovery that the compound of formula (I) is suitable as a fragrance, evoking specific effects in small dosages in combination with other fragrances, particularly fragrances having one or more scent notes selected from the group consisting of the notes fruity, flowery, juicy, fresh, sweet, green, fatty and metallic, preferably one or more scent notes selected from the group consisting of the notes fruity, flowery, juicy, fresh, sweet and green, particularly one or more scent notes selected from the group consisting of the notes fruity and flowery.

Preferably, the use according to the invention relates to a use for imparting, modifying and/or enhancing one or more scent notes selected from the group consisting of the notes fruity, flowery, juicy, fresh, sweet, green, fatty and metallic, preferably one or more scent notes selected from the group consisting of the notes fruity, flowery, juicy, fresh, sweet and green, preferably one or more scent notes selected from the group consisting of the notes fruity and flowery. A preferred embodiment relates to the use of the compound of formula (I) as described herein for imparting one, two, or more, scent notes selected from the group consisting of the notes fruity and flowery.

The fact that the compound of formula (I) to be employed according to the invention is capable of imparting a very complex and varied total sensory impression which, in most cases, may otherwise only be attained by mixtures of several components (such as, for example, essential oils or spice mixtures), is particularly surprising.

In addition to the primary, i.e., scent, properties, the compound of formula (I) also has positive secondary properties, particularly a high substantivity in comparison to fragrances with similar scent properties, a high stability in particular media and compositions, a high abundance, and it is renewable as well.

The context of the preferred use for imparting, modifying and/or enhancing a scent note also includes the finding that the compound of formula (I) may excellently act as what is referred to as a booster (intensifier; enhancer).

The compound of formula (I) to be used in accordance with the invention may also enhance the intensity of a fragrance mixture (fragrance composition), and may lend a finishing olfactory touch to the mixture. Therefore, the compound described herein may be employed in order to lend a fragrance composition more body, freshness, power (of radiation), radiance, lustre, roundness, harmony and/or naturalness.

Furthermore, the compound of formula (I) is suitable as a means for increasing the substantivity and/or retention of a fragrance mixture.

The production of the compound of formula (I) to be used in accordance with the invention may be performed by means of reactions and methods known per se. As the skilled person knows, the compound of formula (I) is an intermediate product from the production process of linalool from pinene. The following FIG. 1 shows a schematic overview of the production process of linalool.

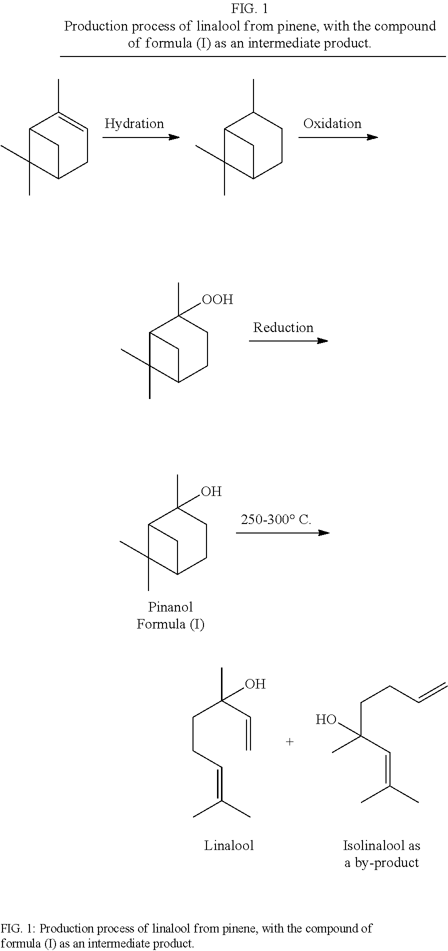

The compound of formula (I) to be employed according to the invention is usually employed in a sensory effective amount within the scope of the use according to the invention, i.e., in a total amount in which it unfolds a sensory effect. Preferably, the compound of formula (I) to be employed according to the invention is employed together with other fragrances. Such fragrance compositions may be produced in the usual manner, for example, by simply mixing or homogeneising the components. These other fragrances may be any other fragrances.

In a preferred embodiment of the invention, the scent note fruity is selected from the group consisting of three elements, pineapple, apple and/or plum. In another preferred embodiment of the invention, the scent note flowery is selected from the group consisting of jasmin, magnolia, peony, lily-of-the-valley, lily, lilac and/or rose.

Further, the present invention relates to a fragrance composition, comprising or consisting of the compound of formula (I)

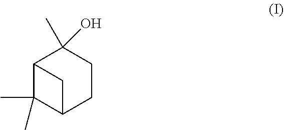

and at least one other fragrance.

Preferably, the fragrance composition comprises or consists of the compound of formula (I) and one, two, three, four, five, six, seven, eight, nine, ten, or more other fragrances.

Preferably, the weight ratio of the total amount of the compounds of formula (I) to the total amount of other fragrances is in the range from 1:1000 to 1:0.1, preferably from 1:1000 to 1:0.5.

In this context, it is preferred that the total amount of the compound of formula (I) is in the range from 0.0001 to 99.9% by weight, preferably 0.001 to 99.5% by weight, particularly preferred 0.01 to 99% by weight, 0.01 to 90% by weight, 0.01 to 50% by weight, 0.01 to 20% by weight, and especially preferred 0.01 to 1.5% by weight, particularly 0.05 to 1.5% by weight, each based on the total weight of the fragrance composition.

Advantageously, the total amount of the compounds of formula (I) in the fragrance composition is selected such that it is contained in a sensory efficient amount which is sufficient to impart and/or to enhance and/or to modify one or more scent notes selected from the group consisting of the notes fruity, flowery, juicy, fresh, sweet, green, fatty and metallic, preferably one or more scent notes selected from the group consisting of the notes fruity, flowery, juicy, fresh, sweet and green.

It is particularly preferred if the one or any one, more, or all of the other fragrances (also) impart, modify and/or enhance, particularly, however, impart, one or more scent notes selected from the group consisting of the notes fruity, flowery, juicy, fresh, sweet, green, fatty and metallic, preferably one or more scent notes selected from the group consisting of the notes fruity, flowery, juicy, fresh, sweet and green.

Examples of fragrances, which may be advantageously combined with a compound of formula (I) within the scope of the present invention, are found, e.g., in S. Arctander, Perfume and Flavor Materials, Vol. I and II, Montclair, N. J. 1969, self-published, or K. Bauer et al., Common Fragrance and Flavor Materials, 4th Edition, Wiley-VCH, Weinheim 2001.

The following are mentioned specifically: extracts from natural raw materials such as essential oils, concretes, absolues, resins, resinoids, balsams, tinctures such as, for example,
Ambratincture; amyris balsamifera oil; angelica seed oil; angelica root oil; anise oil; valerian oil;
Basil oil; tree moss absolue; bay oil; mugwort oil; benzoeresin; bergamot oil;
Beeswax absolue; birch tar; bitter almond oil; savory oil; bucco leaf oil;
Cabreuva oil; cade oil; calmus oil; camphor oil; cananga oil; cardamom oil; cascarilla oil;
Cassia oil; cassie absolue; castoreum absolue; cedar leaf oil; cedar wood oil; cistus oil; citronella oil; lemon oil; copaiba balsam; copaiba balsam oil; coriander oil; costus root oil;
Cumin oil; cypress oil; davana oil; dill herb oil; dill seed oil; eau de brouts absolue;
Oakmoss absolue; elemi oil; tarragon oil; eucalyptus citriodora oil; eucalyptus oil;
Fennel oil; spruce needle oil; galbanum oil; galbanum resin; geranium oil; grapefruit oil;
Guaiac wood oil; gurujun balsam; gurujun balsam oil; helichrysum absolue; helichrysum oil; ginger oil; iris root absolue; iris root oil; jasmin absolue; calamus oil; camomile oil blue;
Camomile oil roman; carrot seed oil; cascarilla oil; pine needle oil; spearmint oil;
Caraway oil; labdanum oil; labdanum absolue; labdanum resin; lavandin absolue;
Lavandin oil; lavender absolue; lavender oil; lemongrass oil; lavage oil; distilled lime oil; pressed lime oil; linaloe oil; litsea cubeba oil; laurel leaf oil; macis oil; marjoram oil; mandarin oil; massoirind oil; mimosa absolue; musk seed oil;
Musk ticture; muscat sage oil; nutmeg oil; myrrh absolue; myrrh oil;
Myrtle oil; clove leaf oil; clove flower oil; neroli oil; olibanum absolue; olibanum oil
Opoponax oil; orange blossom absolue; orange oil; origanum oil; palmarosa oil; patchouli oil;
Perilla oil; Peru balsam oil; parsley leaf oil; parsley seed oil; petitgrain oil; peppermint oil; pepper oil; pimento oil; pine oil; poley oil; rose absolue; rosewood oil; rose oil;
Rosemary oil; sage oil, Dalmatian; sage oil, Spanish; sandalwood oil; celery seed oil; spiked lavender oil; star anise oil; styrax oil; marigold oil; fir needle oil; tea-tree oil; turpentine oil; thyme oil; tolubalsam; tonka-absolue; tuberose absolue; vanilla extract; violet leaf absolue; verbena oil; vetiver oil; juniper berry oil; wine yeast oil; wormwood oil; wintergreen oil; ylang oil; hyssop oil; civet absolue; cinnamon leaf oil; cinnamon bark oil and fractions thereof, or components isolated therefrom;

Single fragrances of the group of carbohydrates such as 3-carene; α-pinene; β-pinene; a-terpinene; γ-terpinene; p-cymene; bisabols; camphene; caryophyllene; cedren; farnesene; limonene; longifolene; myrcene; ocimene; valencene; (E,Z)-1,3,5-undecatriene; styrene; diphenylmethane;
aliphatic alcohols such as hexanol; octanol; 3-octanol; 2,6-dimethylheptanol; 2-methyl-2-heptanal; 2-methyl-2-octanol; (E)-2-hexenol; 1-ectene--3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic aldehydes and their acetals such as hexanal; heptanal; octanal; nonanal; decanal; undecanal; dodecanal; 2-methyloctanal; 2-methylnonanal; (E)-2-hexenal; (Z)-4-heptenal; 2,6-dimethyl--5-heptenal; 10-undecenal; (E)-4-decenal; 2-dodecenal;2,6, 10-trimethyl-9-undecenal; 2,6,10-trimethyl-5,9-undecadienal; heptanaldiethylacetal;
1,1-dimethoxy-2,2,5-trimethyl-4-hexene; citronellyloxyacetaldehyde; 1-(1-methoxy-propoxy)-(E/Z)-3-hexene;
aliphatic ketones and their oximes such as 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
aliphatic sulphur-containing compounds such as 3-methylthiohexanol; methylthiohexyl acetate; 3-mercaptohexanol; 3-mercaptohexyl acetate; 3-mercaptohexyl butyrate; 3-acetylthiohexyl acetate; 1-menthene-8-thiol;
aliphatic nitriles such as 2-nonic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-Dimethyl-6-octenoic acid nitrile;
esters of aliphatic carboxylic acids such as (E)- and (Z)-3-hexenyl formate; ethyl acetoacetate; isoamyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octene-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl-2-methyl pentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxy acetate; methyl 3,7-dimethyl 2,6-octadieneoate; 4-methyl 2-pentyl crotonate;
acyclic terpene alcohols such as geraniol; neral; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5J-octatrien-1-ol; and their formates, acetates, propionates, isobutyrates, butyrates, isovalerates, pentanoates, hexanoates, crotonates, tiglinates, and 3-methyl-2-butenoates;
acyclic terpene aldehydes and ketones such as citronellal; 7-methoxy-3,7-dimethyloctanal; 2,6,10-trimethyl-9-undecenal; geranylacetone; and the dimethyl and diethyl acetals of geranial, neral;
cyclic terpene alcohols such as menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; as well as their formates, acetates, propionates, isobutyrates, butyrates, isovalerates, pentanoates, hexanoates, crotonates, tiglinates, and 3-methyl-2-butenoates;
cyclic terpene aldehydes and ketones such as menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-ionone; beta-damascenone; 1-(2,4,4-trimethyl-2-cyclohexen-1-yl)-2-buten-1-one; 1,3,4,6,7,8a-hexahydro-1,1,5,5-tetramethyl-2H-2,4a-methano-naphthalen-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methylcedrylketone);
cyclic alcohols such as 4-tert.-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
cycloaliphatic alcohols such as alpha,3,3-trimethylcyclohexylmethanol; 1-(4-isopropylcyclohexypethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
cyclic and cycloaliphatic ethers such as cineol; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydro-naphtho[2,1-b]furan; 3a-ethyl-6,6,9a-trimethyldodecahydronaphtho[2,1b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
cyclic and macrocyclic ketones such as 4-tert.-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecanone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;
cycloaliphatic aldehydes such as 2-methyl-4-(2,2,6-trimethyl-cyclohexen-1-yl)-2-butenal; 4-(4-hydroxy-4-methylpentyl)-3-cyclohexene carbaldehyde; 4-(4-methyl-3-penten-1-yl)-3-cyclohexene carbaldehyde;
cycloaliphatic ketones such as 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphtalenylmethylketone; methyl-2,6,10-trimethyl-2,5,9-cyclododecatrienylketone; tert.-butyl-(2,4-dimethyl-3-cyclohexen-1-yl)ketone;
esters of cyclic alcohols such as 2-tert.-butylcyclohexyl acetate; 4-tert.-butylcyclohexyl acetate; 2-tert.-pentylcyclohexyl acetate; 4-tert.-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexa-hydro-5, or 6-indenyl acetate; 4,7-methano-3a,4,5,6,7,7a-hexahydro-5, or 6-indenyl propionate; 4,7-methano-3a,4,5,6,7,7a-hexahydro-5, or 6-indenyl isobutyrate; 4,7-methanooctahydro-5, or 6-indenyl acetate;
esters of cycloaliphatic alcohols such as 1-cyclohexylethyl crotonate;
esters of cycloaliphatic carboxylic acids such as allyl 3-cyclohexyl propionate; allyl cyclohexyloxy acetate; cis and trans methyl dihydrojasmonate; cis and trans methyl jasmonate; methyl 2-hexyl 3-oxocyclopentane carboxylate; ethyl-2-ethyl-6,6-dimethyl-2-cyclohexene carboxylate; ethyl-2,3,6,6-tetramethyl-2-cyclohexene carboxylate; ethyl-2-methyl-1,3-dioxolane-2-acetate;
araliphatic alcohols such as benzyl alcohol; 1-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; methyl-5-phenylpentanol; 3-phenyl-2-propen-1-ol; 4-methoxybenzyl alcohol; 1-(4-Isopropylphenyl)ethanol;
esters of araliphatic alcohols and aliphatic carboxylic acids such as benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
araliphatic ethers such as 2-phenylethyl methyl ether; 2-phenylethyl isoamyl ether; 2-phenylethyl-1-ethoxyethyl ether; phenylacetaldehyde dimethylacetal; phenylacetaldehyde diethylacetal; hydratropic aldehyde dimethylacetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
aromatic and araliphatic aldehydes such as benzaldehyde; phenylacetaldehyde; 3-phenylpropanal; hydratropic aldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-isobutylphenyl)propanal; 3-(4-tert.-butyl-phenyl)propanal; cinnamic aldehyde; alpha-butyl cinnamic aldehyde; alpha-hexyl cinnamic aldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
aromatic and araliphatic ketones such as acetophenone; 4-methylacetophenone; 4-methoxyacetophenone; 4-tert.-butyl-2,6-dimethylaceto-phenone; 4-phenyl-2-butanone; 4-(4-hydroxyphenyl)-2-butanone; 1-(2-naphthalenyl)ethanone; 2-benzofuranylethanone; (3-methyl-2-benzofuranypethanone; benzophenone; 1,1,2,3,3,6-hexamethyl-5-indanylmethylketone; 6-tert.-butyl-1,1-di-methyl-4-indanylmethylketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-aceto-naphthone;
aromatic and araliphatic carboxylic acids and the esters thereof such as benzoic acid; phenylacetic acid; methyl benzoate; ethyl benzoate; hexyl benzoate; benzyl benzoate; methyl phenyl acetate; ethyl phenyl acetate; geranyl phenyl acetate; phenylethyl phenyl acetate; methyl cinnamate; ethyl cinnamate; benzyl cinnamate; phenylethyl cinnamate; cinnamyl cinnamate; allylphenoxyacetate; methyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl-3-phenylglycidate; ethyl-3-methyl-3-phenylglycidate;
nitrogen-containing aromatic compounds such as 2,4,6-trinitro-1,3-dimethyl-5-tert.-butylbenzene; 3,5-dinitro-2,6-dimethyl-4-tert.-butylacetophenone; cinnamic acid nitrile; 3-methyl-5-phenyl-2-pentenoic acid nitrile; 3-methyl-5-phenyl pentanoic acid nitrile; methylanthranilate; methy-N-methylanthranilate; Schiff's bases of methylanthranilate with 7-hydroxy-3,7-dimethyloctanal, 2-methyl-3-(4-tert.-butyl-phenyl)propanal or 2,4-dimethyl-3-cyclohexene carbaldehyde; 6-isopropylquinoline; 6-Isobutylquinoline; 6-sec.-butylquinoline; 2-(3-phenylpropyl)pyridine; indole; skatol; 2-methoxy-3-isopropylpyrazine; 2-isobutyl-3-methoxypyrazine;
phenols, phenyl ethers and phenyl esters such as estragole; anethole; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methyl phenol; 2-ethoxy-5-(1-propenyl)phenol; p-cresyl phenyl acetate;
heterocyclic compounds such as 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-1-pyran-4-one;
lactones such as 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; 1,16-hexadecanolide; 9-hexadecen-1,16-olide; 10-oxa-6-hexadecanolide; 11-oxa-1,16-hexadecanolide; 12-oxa-1,16-hexadecanolide; ethylene-1,12-dodecanedioate; ethylene-1,13-tridecanedioate; 2,3-dihydrocoumarin; octahydrocoumarin.

In a preferred embodiment of the invention, the at least one other fragrance is selected from the group consisting of terpenes, aldehydes, nitriles, esters, lactones, alcohols and mixtures thereof.

In accordance with another preferred embodiment of the fragrance composition of the invention, the compound of formula (I) to be used in accordance with the invention is preferably combined with one or more, particularly preferably two, three, four, five, or more other fragrances which have a flowery and/or fruity scent note.

Accordingly, the present invention also relates to a fragrance composition comprising one, two, three, four, five, or more other fragrances which impart a flowery and/or fruity scent note.

In this case, the compound of formula (I) to be used in accordance with the invention advantageously enhances the scent of the flowery fragrances (at least partially).

Flowery fragrances which may be particularly advantageously combined with the compound of formula (I) to be used in accordance with the invention (particularly in fragrance compositions according to the invention) are preferably selected from the group consisting of:

Hydroxycitronellal, methoxycitronellal, cyclamenaldehyde [2-methyl-3-(4-isopropylphenyl)propanal], 1-(4-isopropyl-cyclohexyl)ethanol (Mugetanol®), 4-tert.-butyl-α-methyldihydrocinnarnic aldehyde (Lilial®), cis-hexahydrocuminyl alcohol (Mayol®), 3-[4-(1,1-dimethylethyl)phenyl]propanal (Bourgeonal®), 2,2-dimethyl-3-(3-methylphenyl)-propanol (Majantol®), 3-methyl-3-(3-methylbenzyl)-butan-2-ol, 2-isobutyl-4-methyltetra-hydro-2H-pyran-4-ol (Florosal), 2-methyl-3-(3,4-methylenedioxyphenyl)propanal (Heliofolal®), 4-(4-hydroxy-4-methylpentyl)-3-cyclohexene carbaldehyde (Lyral®), 4-(octahydro-4,7-methano-5H-inden-5-ylidene-butanal (Dupical®), vernaldehyde, ethyl-2-methylbutanoate, 4-(4-methyl-3-penten-1-yl)-3-cyclohexene carbaldehyde (Vertomugal®), octahydro-5-(4-methoxybutyl idene)-4,7-methano-1H-indene (Mugoflor®), 2,6-dimethyl-2-heptanal (Freesiol®), 1-ethyl-1-methyl-3-phenylpropanol (Phemec®), 2,2-dimethyl-3-phenyl-1-propanol (Muguet alcohol), profarnesol, dihydrofarnesol, farnesol, nerolidol, hydroxycitronellaldimethylacetal, hexylbenzoate, geraniol, nerol, linalool, tetrahydrogeraniol, tetrahydrolinalool, ethyllinalool, geranyltiglinate, phenethyl alcohol (2-phenylethyl alcohol), 3,7-dimethyloct-6-en-1-ol (citronellol), rose oxide, 2-methyl-5-phenylpentanol (rosaphene), 3-methyl-5-phenylpentanol (phenoxanol), methyldihydrojasmonate (Hedion®, Hedione® high cis), 2-heptylcyclopentanone (Projasmon P), cis-jasmon, dihydrojasmon, cinnamon alcohol (3-phenyl-2-propen-1-01), dihydrocinnamon alcohol (3-phenylpropanol), 2-methyl-4-phenyl-1,3-dioxolane (Jacinthaflor@), benzyl acetate, benzyl salicylate and dihydromyrcenol (2,6-dimethyl-7-octen-2-ol).

In addition, the compound of formula (I) to be used in accordance with the invention is advantageously suitable to enhance particularly fruity fragrances with respect to their scent.

Fruity fragrances with which the compound of formula (I) to be used according to the invention can be advantageously combined, and which are therefore particularly preferred (other) fragrances of fragrance compositions according to the invention, are preferably selected from the group consisting of:

Benzyl acetate, ethyl (2,4-dimethyl-1,3-dioxolan-2-yl) acetate (Fragolane), ethyl-2-cyclopentenyl acetate (Sultanene), 1-cyclohexylethyl (E)-but-2-enoate (Datilat), 2-methyl-butyric acid ethyl ester, 4-(p-hydroxyphenyl)-2-butanone, ethyl-3-methyl-3-phenylglycidate, butyric acid isoamyl ester, acetic acid isoamyl ester, acetic acid n-butyl ester, butyric acid ethyl ester, 3-methyl-butyric acid ethyl ester, n-hexanoic acid ethyl ester, n-hexanoic acid allyl ester, ethyl-2-trans-4-cis-decadienoate, 1,1-dimethoxy-2,2,5-trimethyl-4-hexane, 2,6-dimethyl-5-hepten-1-al, gamma-undecalactone, gamma-nonalactone, hexanal, 3Z-hexenal, n-decanal, n-dodecanal, citral, vanillin, ethylvanillin, maltol, ethylmaltol, 4-methylpentan-2-yl (E)-but-2-enoate (Frutinat), (2-cyclopentylcyclopentyl) (E)-but-2-enoate (Pyroprunat) and mixtures thereof.

In a preferred embodiment of the invention, the at least one other fragrance is selected from the group consisting of ethyl-2-cyclopentenylacetate, 1-cyclohexylethyl (E)-but-2-enoate, ethyl 2-methylbutanoate, ethyl (2,4-dimethyl-1,3-dioxolan-2-yl)acetate, 4-methylpentan-2-yl(E)-but-2-enoate, (2-cyclopentylcyclopentyl)(E)-but-2-enoate, phenylethyl alcohol, 5-(3-methylphenyl)pentan-1-ol, benzylacetate, methyl-3-oxo-2-pentylcyclopentan acetate, 1-(4-propan-2-ylcyclohexyl)ethanol, 3,7-dimethyloct-6-en-1-ol, phenoxanol, benzylsalicylate, 2,2-dimethyl-3-(3-methylphenyl)propan-1-ol and/or mixtures thereof.

Fragrance compositions according to the invention which contain the compound of formula (I) may be in liquid form, undiluted or diluted with a solvent or are advantageously employed for perfuming. Preferred solvents for this purpose include ethanol, isopropanol, diethylene glycol monoethyl ether, glycerol, propylene glycol, 1,2-butylene glycol, dipropylene glycol, diethyl phthalate, triethyl citrate, isopropyl myristate, triacetine and diacetine.

Furthermore, fragrance compositions according to the invention may be adsorbed on a carrier material which provides both a fine distribution of the fragrances in the product and a controlled release during application. Suitable carriers may be porous inorganic materials such as light sulphate, silica gels, zeolites, gypsums, clays, clay granules, aerated concrete, etc. or organic materials such as wood, cellulose-based materials, sugar, dextrins (e.g., maltodextrin), or plastics such as PVC, polyvinyl acetates or polyurethanes. The resulting combination of a composition according to the invention and a carrier material is also to be understood as a fragrance composition according to the invention or may be present as an article according to the invention (as described herein below).

Fragrance compositions according to the invention may also be microencapsulated, spray-dried, or may be present as inclusion complexes or as extrusion products and in the case of a fragrance composition, may be added in this form, e.g., to a product to be perfumed.

Optionally, the properties of such modified compositions or articles may be further optimised by what is referred to as “coating” with suitable materials with regard to a more targeted release of fragrance, preferably using wax-like plastics such as polyvinyl alcohol. The resulting products in turn represent articles according to the invention.

A further aspect of the present invention relates to the use of a fragrance composition for imparting, modifying and/or enhancing one or more scent notes selected from the group consisting of the notes fruity, flowery, juicy, fresh, sweet, green, fatty and metallic, preferably one or more scent notes selected from the group consisting of the notes fruity, flowery, juicy, fresh, sweet and green, In a particularly preferred form, the scent notes are selected from the notes fruity and flowery. With respect to preferred embodiments, what has been mentioned above in connection with a use in accordance with the invention or a fragrance composition in accordance with the invention shall apply mutatis mutandis.

Advantageously, fragrance compositions according to the invention may be used in concentrated form, in solutions, or in the modified form described above for the production of perfumed articles according to the invention such as, for example, perfume extracts, eau de parfums, eau de toilettes, shaving lotions, eau de colognes, pre-shave products, splash colognes and perfumed refreshing wipes, as well as the perfuming of acidic, alkaline and neutral detergents, such as floor cleaners, window glass cleaners, dishwashing detergents, bathroom and sanitary cleaners, scouring milk, solid and liquid WC cleaners, powder and foam carpet cleaners, textile fresheners, ironing aids, liquid laundry detergents, powder laundry detergents, laundry pretreatment agents such as bleaching agents, soaking agents and stain removers, fabric softeners, washing soaps, washing tablets, disinfectants, surface disinfectants and air fresheners in liquid or gel form, or a form applied on a solid carrier, aerosol sprays, waxes and polishes such as furniture polishes, floor waxes, shoe polishes and body care products such as solid and liquid soaps, shower gels, shampoos, shaving soaps, shaving foams, bath oils, cosmetic emulsions of oil-in-water, water-in-oil and water-in-oil-in-water types such as skin creams and lotions, facial creams and lotions, sun protection creams and lotions, after-sun creams and lotions, hand creams and lotions, foot creams and lotions, depilatory creams and lotions, after-shave creams and lotions, tanning creams and lotions, hair care products such as hair sprays, hair gels, styling hair lotions, hair conditioners, permanent and semi-permanent hair dyes, hair deformers such as cold waves and hair straightening agents, hair toners, hair creams and lotions, deodorants and antiperspirants such as underarm sprays, roll-ons, deodorant sticks, deodorant creams, decorative cosmetic products such as eye shadows, nail varnishes, make-ups, lipsticks, mascara as well as candles, lamp oils, incense sticks, insecticides, repellents, and fuels.

Of course, fragrance compositions according to the invention may also be part of the most diverse cosmetic compositions or household chemical compositions.

A further aspect of the invention concerns a process for imparting, enhancing and/or modifying one or more scent notes selected from the group consisting of the notes fruity, flowery, juicy, fresh, sweet, green, fatty and metallic, preferably one or more scent notes selected from the group consisting of the notes fruity, flowery, juicy, fresh, sweet and green, particularly preferred one or more scent notes selected from the group consisting of the notes fruity and flowery, comprising or consisting of the following steps
(a) Providing the compound of formula (I)

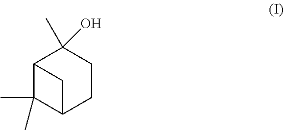

(b) Providing at least one other fragrance,
(c) Adding the compound of formula (I) to the at least one other fragrance in a sensory effective amount which is sufficient to impart and/or enhance and/or modify one or more scent notes selected from the group consisting of the notes fruity, flowery, juicy, fresh, sweet, green, fatty and metallic, preferably one or more scent notes selected from the group consisting of the notes fruity, flowery, juicy, fresh, sweet and green.

In a preferred embodiment of the invention the at least one other fragrance has one or more scent notes selected from the group consisting of the notes fruity, flowery, juicy, fresh, sweet, green, fatty and metallic.

A further aspect of the present invention relates to a perfumed article, comprising or consisting of
(i) a fragrance composition according to the invention, as defined above;
(ii) at least one other fragrance, and
(iii) one or more other components, preferably one or more adjuvants and/or active ingredients,

In a preferred embodiment, the article is selected from cosmetic, hygiene and/or household articles. With respect to preferred embodiments, what has been mentioned above in connection with a use in accordance with the invention or a fragrance composition in accordance with the invention shall apply mutatis mutandis.

Advantageously, a perfumed article according to the invention is selected from the group consisting of washing and cleaning agents, personal care and hair care products, and household products, preferably from the group consisting of perfume extracts, eau de parfums, eau de toilettes, shaving lotions, eau de colognes, pre-shave products, splash colognes and perfumed refreshing wipes, acidic, alkaline and neutral detergents, textile fresheners, ironing aids, liquid laundry detergents, powder laundry detergents, laundry pretreatment agents, fabric softeners, washing soaps, washing tablets, disinfectants, surface disinfectants, air fresheners, aerosol sprays, waxes and polishes, body care products, hand creams and lotions, foot creams and lotions, depilatory creams and lotions, after-shave creams and lotions, tanning creams and lotions, hair care products, deodorants and antiperspirants, decorative cosmetic products, candles, lamp oils, incense sticks, insecticides, repellents, and fuels,

In addition, for a perfumed article according to the invention it is preferred that component (i) is contained in a sensory effective amount which is sufficient for a consumer to perceive one or more scent notes selected from the group consisting of the notes fruity, flowery, juicy, fresh, sweet, green.

It is also preferred that the total amount of the compound of formula (I) in the perfumed article is in the range from 0.00001 to 10% by weight, preferably 0.0001 to 5% by weight, particularly preferred 0.001 to 2% by weight, more preferably 0.005 to 1% by weight, based on the total weight of the article.

The additives, adjuvants and/or active ingredients described above are preferably not fragrances. They may be, for example, preservatives, anti-bacterial agents, chelating agents, cleaning agents, emulsifiers, fats, etc., and principally all substances that are employed as additives, adjuvants and/or active ingredients in the cosmetic industry, specifically in fragrance compositions and in household chemical compositions.

In the following, the invention is further characterised by means of examples of embodiment.

### EXAMPLES

### Example 1: Description of the Scent of Preferred Fragrances After Adding 2,6 1 6-trimethyl-norpinan-2-ol (Pinanol)

TABLE 1
Description of the scent with the addition of Pinanol. All indications of amounts are weight percent indications

| | | | | Description of the |
|---|---|---|---|---|
| | | | | scent in comparison |
| | | | Dosage of | with the scent of |
| No.: | Fragrance | Structure | Pinanol | the pure fragrance |
| 1 | Sultanene ethyl-2-cyclopentenyl acetate | 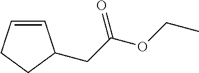 | 1% | More juicy More natural pineapple note |

| | | | | |
|---|---|---|---|---|
| 2 | Datilate 1-cyclohexylethyl (E)- but-2-enoate | 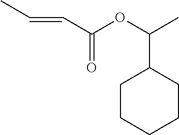 | 0.2% | The scent has a more powerful effect More fruity, more juicy, and more fresh |

| | | | | |
|---|---|---|---|---|
| 3 | ethyl 2-methyl butanoate | 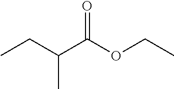 | 0.2% | Yields a wine yeast note and a lilac note More fruity apple note More intense |
| 4 | Fragolane Ethyl (2,4-dimethyl-1,3- dioxolan-2-yl) acetate | 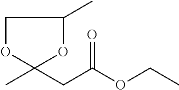 | 0.3% | Greener (more grassy) More juicy Yields longlastingness |

| | | | | |
|---|---|---|---|---|
| 5 | Frutinat 4-methylpentan-2-yl (E)-but-2-enoate | 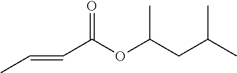 | 0.3% | Enhances the apple note |
| 6 | Pyroprunat (2- cyclopentylcyclopentyl) (E)-but-2-enoate | 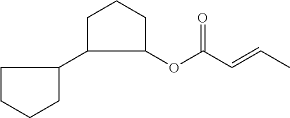 [SMILES: C/C=C/C(=O)OC1CCCC1C1CCCC1] | 0.3% | Yields a more intense plum note and a damascenone note |
| 7 | Phenylethyl alcohol | 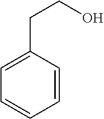 | 0.1% | Enhances the flowery notes Yields intensity and body |
| 8 | Rosaphen 5-(3- methylphenyl)pentan- 1-ol | 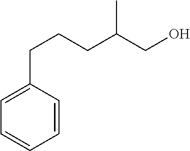 | 0.2% | More natural rose note Yields a magnolan effect More intense |
| 9 | Benzyl acetate | 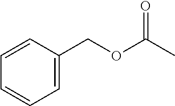 | 0.5% | More natural jasmin note More intense Enhances the fruity note |
| 10 | Hedion Methyl-3-oxo-2- pentylcyclopentane acetate | 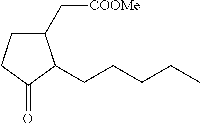 [SMILES: CCCCCC1C(=O)CCC1CC(=O)OC] | 0.1% | More flowery Yields a more natural jasmin note |
| 11 | Mugetanol 1-(4-propan-2- ylcyclohexyl)ethanol | 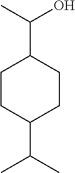 | 0.3% | More intense sweet note Reduces the technical character Yields a more natural lily-of-the- valley note |
| 12 | Citronellol 3,7-Dimethyloct-6-en- 1-ol | 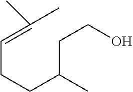 | 0.2% | Enhances the green note Yields a lemon grass note |
| 13 | Phenoxanol | 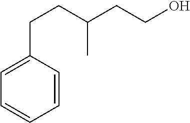 | 0.1% | More fresh More natural rose note Enhances the citronellol note |
| 14 | Benzyl salicylate | 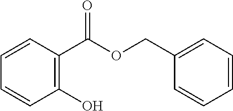 [SMILES: O=C(OCc1ccccc1)c1ccccc1O] | 0.025% | Enhances the salicylate effect More intense Rose effect |
| 15 | Majantol 2,2-dimethyl-3-(3- methylphenyl)propan- 1-ol | 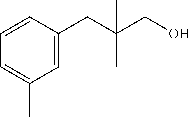 | 0.3% | More intense lilac and freesia notes Lilial facettes Reduces the unpleasant technical head note |

### Example 2: Perfume Oils With Flowery Notes

Peony and jasmine

| | |
|---|---|
| Pinanol (Formula I) | 0.1% |
| Neocyclocitral 1% DPG | 0.7% |
| Magnolan | 3% |
| Dihydro Myrcenyl Acetate | 2% |
| Hivernal Neo 10% DPG | 0.5% |
| Lillybelle ® 10% DPG | 0.2% |
| Mugenal 50% DPG | 0.2% |
| Ethyl Linalool | 10% |
| Terpineol Alpha | 0.1% |
| Phenylethyl Alcohol | 50% |
| Citronellol 950 | 1% |
| Geraniol Supra | 0.8% |
| Phenoxanol | 0.5% |
| Benzyl Acetate | 0.2% |
| Hedione | 10% |
| Benzyl Salicylate | 1.5% |
| Eugenol | 0.1% |
| Elemi Oil | 0.2% |
| Cuminic Alcohol | 0.08% |
| Dipropylene Glycol | 18.82% |

Jasmine

| | |
|---|---|
| Pinanol Formula (I) | 0.1% |
| Stemone 10% DPG | 0.3% |
| Methyl Naphtyl Ketone Beta Cryst. 10% DPG | 0.5% |
| Aldehyde C14 so-called 10% DPG | 0.5% |
| Aldehyde C16 so-called 10% DPG | 0.3% |
| Linalool | 7% |
| Cresol Para 10% DPG | 0.4% |
| Cresyl Phenyl Acetate Para | 0.3% |
| Benzyl Acetate | 25% |
| Hedione HC/30 | 10% |
| Hexyl Cinnamic Aldehyde Alpha | 20% |
| Jasmolactone CNC | 0.2% |
| Jasmone Cis | 2% |
| Eugenol Nat. | 0.3% |
| Indolene 50 Castor Oil | 2.5% |
| Dipropylene Glycol | 30.6% |

According to the statements of a trained panel, the addition of the compound of formula (I) enhances the flowery notes, rounds off the mixtures and also creates naturalness.

### Example 3: Perfume Oils With Fruity Notes

Pineapple

| | |
|---|---|
| Pinanol (Formula I) | 0.1% |
| Dynascone 1% DPG | 0.3% |
| Allyl Amyl Glycolate | 0.2% |
| Amyl Butyrate N/ISO | 1% |
| Ethyl Caproate | 5% |
| Ethyl Heptoate | 15% |
| Methyl Caproate | 2% |
| Diethyl Malonate | 1% |
| Ethyl Methyl Butyrate-2 | 0.5% |
| Allyl Caproate | 10% |
| Allyl Cyclohexyl Propionate | 20% |
| Cycloannanat/Corps 571 | 0.1% |
| Ethyl Maltol | 0.8% |
| Herbaflorat | 0.5% |
| Dipropylene Glycol | 43.5% |

Blueberry

| | |
|---|---|
| Pinanol Formula (I) | 0.1% |
| Hexenol Cis-3 | 4% |
| Hexenyl Isovalerate Cis-3 | 4% |
| Eucalyptol Nat 1% DPG | 0.5% |
| Prenyl Acetate | 1% |
| Ethyl Isovalerate | 14% |
| Isovaleric Acid 1% DPG | 0.2% |
| Ethyl Caproate 10% DPG | 1% |
| Dodecalactone Gamma | 2.5% |
| Davana Oil F. Perf. | 1% |
| Cassis Abs. Burgundy 10% DPG | 1% |
| Frambinon ® 10% DPG | 0.3% |
| Maltol 1% DPG | 2% |
| Ethyl Linalool | 7.5% |
| Dimethyl Benzyl Carbinyl Butyrate | 4% |
| Phenylethyl Isovalerate | 2% |
| Geraniol Supra | 8% |
| Damascenone 10% DPG | 1% |
| Sandal Mysore Core 1% DPG | 0.1% |
| Helvetolide | 0.5% |
| Dipropylene Glycol | 45.3% |

According to the statements of a trained panel, the addition of the compound of formula (I) enhances the fruity notes while the mixture obtains a natural note. In addition, the compound of formula (I) creates a juicy and fresh note.

1. A method of imparting a fragrance, comprising the step of applying a compound of formula (I) 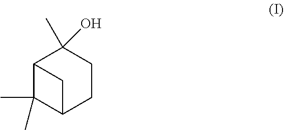
as the fragrance.

2. The method of claim 1, comprising the additional step of imparting, modifying and/or enhancing one or more scent notes selected from the group consisting of the notes fruity, flowery, juicy, fresh, sweet, green, fatty, metallic, and mixtures thereof, preferably one or more scent notes selected from the group consisting of the notes fruity, flowery, juicy, fresh, sweet, green, and mixtures thereof.

3. The method of claim 2, wherein the scent note fruity is selected from pineapple, apple, and/or plum.

4. The method of claim 2, wherein the scent note flowery is selected from jasmin, magnolia, peony, lily of the valley, lily, lilac and/or rose.

5. Fragrance composition, comprising the compound of formula (I) 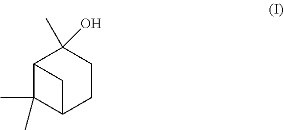
and at least one other fragrance.

6. The fragrance composition of claim 5, wherein the weight ratio of the total amount of the compound of formula (I) to the total amount of other fragrance is in the range from 1:1000 to 1:0.1.

7. The fragrance composition of claim 5, wherein the total amount of the compound of formula (I) is in the range from 0.0001 to 99.9% by weight, based on the total weight of the fragrance composition.

8. The fragrance composition of claim 5, wherein the total amount of the compound of formula (I) is contained in the fragrance composition in a sensory effective amount sufficient to impart and/or enhance and/or modify one or more scent notes selected from the group consisting of the notes fruity, flowery, juicy, fresh, sweet, green, fatty, metallic, and mixtures thereof, preferably one or more scent notes selected from the group consisting of the notes fruity, flowery, juicy, fresh, sweet, green, and mixtures thereof.

9. The fragrance composition of claim 5, wherein the at least one other fragrance is selected from the group consisting of terpenes, aldehydes, nitriles, esters, lactones, alcohols, and mixtures thereof.

10. The fragrance composition of claim 5, wherein the at least one other fragrance is selected from the group consisting of ethyl-2-cyclopentenylacetate, 1-cyclohexylethyl (E)-but-2-enoate, ethyl 2-methylbutanoate, ethyl (2,4-dimethyl-1,3-dioxolan-2-yl)acetate, 4-methylpentan-2-yl(E)-but-2-enoate, (2-cyclopentylcyclopentyl)(E)-but-2-enoate, phenylethyl alcohol, 5-(3-methylphenyl)pentan-1-ol, benzylacetate, methyl-3-oxo-2-pentylcyclopentan acetate, 1-(4-propan-2-ylcyclohexyl)ethanol, 3,7-dimethyloct-6-en-1-ol, phenoxanol, benzylsalicylate, 2,2-dimethyl-3-(3-methylphenyl)propan-1-ol, and and/or mixtures thereof.

11. A method comprising the step of imparting, modifying and/or enhancing one or more scent notes selected from the group consisting of the notes fruity, flowery, juicy, fresh, sweet, green, fatty, metallic, and mixtures thereof, preferably one or more scent notes selected from the group consisting of the notes fruity, flowery, juicy, fresh, sweet, green, and mixtures thereof, by applying the fragrance composition of claim 5.

12. Process for imparting, enhancing and/or modifying one or more scent notes selected from the group consisting of the notes fruity, flowery, juicy, fresh, sweet, green, fatty, metallic, and mixtures thereof, preferably one or more scent notes selected from the group consisting of the notes fruity, flowery, juicy, fresh, sweet, green, and mixtures thereof, comprising of the following steps:
(a) Providing the compound of formula (I) 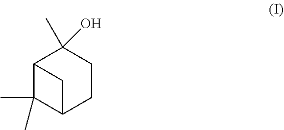
(b) Providing at least one other fragrance, and
(c) Adding the compound of formula (I) to the at least one other fragrance in a sensory effective amount which is sufficient to impart and/or enhance and/or modify one or more scent notes selected from the group consisting of the notes fruity, flowery, juicy, fresh, sweet, green, fatty, metallic, and mixtures thereof, preferably one or more scent notes selected from the group consisting of the notes fruity, flowery, juicy, fresh, sweet, green, and mixtures thereof.

13. The process of claim 12, wherein the at least one other fragrance has one or more scent notes selected from the group consisting of the notes fruity, flowery, juicy, fresh, sweet, green, fatty, metallic, and mixtures thereof.

14. Perfumed article, comprising
(i) a fragrance composition of claim 5,
(ii) at least one other fragrance, and
(iii) one or more other components, preferably one or more adjuvants and/or effective ingredients.

15. The perfumed article of claim 14, wherein the article is selected from the group consisting of cosmetic, personal care and/or household articles.
